(19) United States
(12) Patent Application Publication
(10) Pub. No.: US 2023/0019615 A1
(43) Pub. Date: Jan. 19, 2023

(54) COMPOSITIONS AND METHODS FOR STYLING HAIR

(71) Applicant: L'Oreal, Paris (FR)

(72) Inventors: Vibha K. SHAH, Branchburg, NJ (US); Rachel FEREBEE MAHER, Pittsburgh, PA (US); Allison PERNER, Metuchen, NJ (US)

(21) Appl. No.: 17/853,071

(22) Filed: Jun. 29, 2022

Related U.S. Application Data
(60) Provisional application No. 63/217,203, filed on Jun. 30, 2021.

(30) Foreign Application Priority Data
Sep. 20, 2021 (FR) 2109867

(51) Int. Cl. A61K 8/368; A61K 8/365; A61K 8/73; A61K 8/34; A61K 8/37; A61K 8/898; A61K 8/41; A61Q 5/06

(52) U.S. Cl. CPC A61K 8/368; A61K 8/365; A61K 8/738; A61K 8/342; A61K 8/37; A61K 8/898; A61K 8/416; A61Q 5/06; A61K 2800/5422; A61K 2800/30

(57) ABSTRACT

The disclosure relates to compositions comprising (a) at least one polyphenol; (b) at least one nonionic polymer; (c) at least one cationic surfactant; (d) at least one emulsifier; and (e) at least one solvent. The compositions optionally further comprise at least one silicone compound and/or at least one oil. The compositions can be used for styling hair, for example temporarily straightening the hair and/or for reducing frizz of hair. The disclosure also relates to methods of using the compositions to style hair, straighten hair, and/or reduce frizz of hair.

### TECHNICAL FIELD

The present disclosure relates to compositions and methods for styling hair, such as curly or wavy hair. The compositions provide temporary straightening properties to the hair. The disclosure also relates to methods of using the compositions to style the hair.

### BACKGROUND

Consumers with curly or wavy hair often desire to temporarily straighten their hair for a change in appearance. In addition, reducing the curls of very curly hair may increase the manageability and ease of styling of such hair.

Traditional hair-straightening products generally include heavy galenics or oils, which may leave an undesirable greasy or oily feeling. Products that provide hold, such as styling gels, can also give straightening effects, but these products typically flake on the hair. In addition, many hair straightening products involve heat or chemical treatments that may cause damage to the hair fibers and/or irritate the scalp.

As such, consumers desire new and improved compositions that can provide straightening effects to curly or wavy hair, and at the same time impart various additional advantageous properties to the hair such as softness, smoothness, shine, and/or frizz control.

It has now surprisingly been found that compositions comprising a particular combination of components as described herein are able to temporarily straighten curly or wavy hair without the use of heat or harsh chemicals, while at the same time delivering additional advantageous sensorial benefits to the hair.

### SUMMARY

The present disclosure relates to compositions for styling hair, such as curly or wavy hair. The compositions may provide beneficial effects such as straightening the hair and/or one or more of softness, smoothness, shine, volume control, and/or frizz control to the hair. The compositions are optionally leave-in compositions, and may, in various embodiments, be in the form of lotion or cream emulsions.

The hair styling compositions may comprise, in certain embodiments: (a) at least one polyphenol; (b) at least one nonionic polysaccharide polymer; (c) at least one cationic surfactant; (d) at least one emulsifier; and (e) at least one solvent. Optionally, the hair styling compositions may further comprise at least one silicone compound and/or at least one oil and/or at least one cyclodextrin.

The at least one polyphenol may be chosen from flavonoids and non-flavonoids, as well as mixtures thereof, and in some embodiments comprises, consists essentially or, or consists of one or more flavonoids. In a one exemplary and non-limiting embodiment, the flavanoid comprises, consists essentially or, or consists of tannic acid. The at least one polyphenol may be present in an amount ranging from about 0.01% to about 10% by weight, relative to the total weight of the composition, such as, for example, about 0.1% to about 8%, about 0.5% to about 5%, or about 1% to about 3%. For example, the composition may comprise tannic acid, present in an amount ranging from about 0.01% to about 10% by weight, relative to the total weight of the composition, such as, for example, about 0.1% to about 8%, about 0.5% to about 5%, or about 1% to about 3%.

The at least one nonionic polysaccharide polymer may be chosen from nonionic C1-C3 alkyl guars, nonionic C1-C3 hydroxyalkyl guars, glucans, modified or non-modified starches, dextrans, celluloses and derivatives thereof, or mixtures thereof, and may, in one exemplary and non-limiting embodiment, comprise, consist essentially of, or consist of one or more glucans, for example sclerotium gum. The at least one nonionic polysaccharide polymer may be present in an amount ranging from about 0.01% to about 5% by weight, relative to the total weight of the composition, such as, for example, about 0.05% to about 3%, about 0.1% to about 2%, or about 0.1% to about 1%. For example, the composition may comprise sclerotium gum, present in an amount ranging from about 0.01% to about 5% by weight, relative to the total weight of the composition, such as, for example, about 0.05% to about 3%, about 0.1% to about 2%, or about 0.1% to about 1%.

The at least one cationic surfactant may be chosen from amidoamine compounds, monoalkyl quaternary amines, dialkyl quaternary amines, or polyquaternium compounds or salts thereof, fatty alkylamines, polyoxyalkylenated primary, secondary, or tertiary fatty amine salts, or quaternary ammonium salts. The at least one cationic surfactant may include at least one tetraalkylammonium halide. In some exemplary and non-limiting embodiments, the at least one cationic surfactant may comprise, consist essentially of, or consist of behentrimonium chloride, cetrimonium chloride, or a mixture thereof. The at least one cationic surfactant can be present in the composition in an amount ranging from about 0.01% to about 10% by weight, relative to the total weight of the composition, such as from about 0.05% to about 5%, about 0.1% to about 3%, or about 0.1% to about 1%.

The at least one emulsifier may be chosen from fatty alcohols, esters, or mixtures thereof. In some embodiments, the at least one emulsifier comprises at least one fatty alcohol and at least one ester.

The fatty alcohols may be chosen from, for example, C6-C30 alkoxylated or non-alkoxylated, saturated or unsaturated, linear or branched, fatty alcohols, and may be liquid or solid. In an exemplary and non-limiting embodiment, the fatty alcohols may be chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, derivatives thereof, or mixtures thereof. The fatty alcohols may be present in an amount ranging from about 0.1% to about 10% by weight, relative to the total weight of the composition, such as from about 1% to about 10%, about 2% to about 8%, or about 3% to about 7%.

The esters may be chosen from glyceryl esters or non-glyceryl esters. Non-glyceryl esters may be chosen from, by way of example, isopropyl esters, cetyl esters, or a mixture thereof. The esters may be present in an amount ranging from about 0.01% to about 5% by weight, relative to the total weight of the composition, such as from about 0.05% to about 3%, or about 0.1% to about 2%.

The solvent comprises water and optionally at least one non-aqueous solvent, and may be present in an amount ranging from about 50% to about 99%, such as about 60% to about 98%, or about 70% to about 95% by weight, relative to the total weight of the composition. In one exemplary and non-limiting embodiment, the solvent consists of or consists essentially of water, with the solvent comprising less than about 10%, less than about 5%, or less than about 3% by weight, relative to the total weight of the composition, of non-aqueous solvents.

If present, the at least one silicone compound may be chosen from dimethicone, dimethicone copolymers, amino functional silicones, or mixtures thereof. In one exemplary and non-limiting embodiment, the silicone compound comprises, consists essentially of, or consists of amodimethicone. The silicone compound(s) may be present in an amount ranging from about 0.1% to about 10% by weight, relative to the total weight of the composition, such as from about 0.5% to about 6%, or about 1% to about 5%.

The at least one oil, if present, can, for example, be chosen from hydrocarbon-based oils of plant origin and/or vegetable origin, or glyceride triesters. By way of example, coconut or olive oil can be included. The oil may be present in an amount ranging from about 0.1% to about 10% by weight, relative to the total weight of the composition, such as from about 0.5% to about 5%, or about 1% to about 3%.

In some embodiments, the compositions may optionally further comprise at least one cyclodextrin.

In further embodiments, hair styling compositions according to the disclosure may comprise (a) at least one flavonoid; (b) at least one glucan; (c) at least one cationic surfactant; (d) at least one emulsifier; (e) water; (f) optionally at least one silicone compound; and (g) optionally at least one oil. These compositions may optionally further comprise at least one cyclodextrin.

In certain embodiments, hair styling compositions according to the disclosure may comprise (a) tannic acid; (b) at least one nonionic polysaccharide polymer; (c) at least one cationic surfactant; (d) an emulsifier component comprising at least one fatty alcohol and at least one ester; (e) water, present in an amount of at least 50% by weight, relative to the total weight of the hair styling composition; (f) optionally, at least one amino functional silicone; and (g) optionally, at least one oil.

In some embodiments, hair styling compositions according to the disclosure may comprise (a) tannic acid; (b) sclerotium gum; (c) at least one cationic surfactant; (d) an emulsifier component comprising at least one fatty alcohol and at least one ester; (e) water, present in an amount of at least 50% by weight, relative to the total weight of the hair styling composition; (f) at least one amino functional silicone; and (g) at least one oil. These compositions may further comprise at least one cyclodextrin.

In further embodiments, hair styling compositions according to the disclosure may comprise (a) tannic acid, present in an amount ranging from about 0.5% to about 5%; (b) at least one nonionic polysaccharide polymer, present in an amount ranging from about 0.1% to about 1%; (c) at least one cationic surfactant, present in an amount ranging from about 0.1% to about 1.5%; (d) an emulsifier component comprising: (i) from about 1% to about 8% of at least one fatty alcohol, and (ii) from about 0.1% to about 2% of at least one ester; (e) water, present in an amount of at least 50%; (f) at least one amino functional silicone, present in an amount ranging from about 1% to about 5%; and (g) at least one oil, wherein all amounts are by weight, relative to the total weight of the composition. These compositions may optionally further comprise at least one cyclodextrin, present in an amount ranging from about 0.01% to about 20% by weight, relative to the total weight of the composition.

In yet further embodiments, hair styling compositions according to the disclosure may comprise (a) tannic acid; (b) sclerotium gum; (c) at least one cationic surfactant comprising behentrimonium chloride, cetrimonium chloride, or mixtures thereof; (d) at least one emulsifier comprising: (i) cetearyl alcohol, and (ii) cetyl esters; (e) water; (f) amodimethicone; and (g) at least one oil. These compositions may optionally further comprise at least one cyclodextrin, such as β-cyclodextrin.

The disclosure further relates to methods of using the compositions, such as methods of styling the hair, for example methods of straightening the hair and/or methods of reducing frizz, wherein the methods comprise applying the composition to wet, damp, or dry hair.

For example, in various embodiments, the disclosure relates to methods of styling the hair, methods of straightening the hair, and/or methods of reducing frizz of the hair, comprising applying to the hair a composition comprising: (a) at least one polyphenol; (b) at least one nonionic polysaccharide polymer; (c) at least one cationic surfactant; (d) at least one emulsifier; (e) at least one solvent comprising water; (f) optionally, at least one silicone compound; and (g) optionally, at least one oil. In various methods of styling the hair, straightening the hair, and/or reducing frizz of the hair, the at least one polyphenol is chosen from flavonoid compounds, for example tannic acid, which may be present in an amount ranging from about 0.01% to about 10% by weight, such as from about 0.1% to about 8%, about 0.5% to about 5%, or about 1% to about 3% by weight, relative to the total weight of the composition. In various embodiments, the at least one nonionic polysaccharide polymer that can be used in compositions for styling the hair, straightening the hair, and/or reducing frizz of the hair include, for example, nonionic C1-C3 alkyl guars, nonionic C1-C3 hydroxyalkyl guars, glucans, modified or non-modified starches, dextrans, celluloses and derivatives thereof, or mixtures thereof, for example sclerotium gum, which may be present in an amount ranging from about 0.01% to about 5% by weight, relative to the total weight of the composition, such as from about 0.05% to about 3%, about 0.1% to about 2%, or about 0.1% to about 1%. The at least one cationic surfactant used in the methods for styling the hair, straightening the hair, and/or reducing frizz of the hair can be chosen from, for example, amidoamine compounds, monoalkyl quaternary amines, dialkyl quaternary amines, or polyquaternium compounds or salts thereof, fatty alkylamines, polyoxyalkylenated primary, secondary, or tertiary fatty amine salts, quaternary ammonium salts, or mixtures thereof, and may be present in an amount ranging from about 0.01% to about 10% by weight, relative to the total weight of the composition, such as from about 0.05% to about 5%, about 0.1% to about 3%, or about 0.1% to about 1%. The at least one emulsifier may be chosen from, for example, fatty alcohols, esters, or mixtures thereof, and in some embodiments, the at least one emulsifier comprises at least one fatty alcohol present in an amount ranging from about 0.1% to about 10% by weight, relative to the total weight of the composition, such as from about 1% to about 10%, about 2% to about 8%, or about 3% to about 7%, and/or at least one ester present in an amount ranging from about 0.01% to about 5% by weight, relative to the total weight of the composition, such as from about 0.05% to about 3%, or about 0.1% to about 2%. The solvent in the composition useful in the methods of styling, straightening, and/or reducing frizz of the hair comprises water and optionally at least one non-aqueous solvent, and may be present in an amount ranging from about 50% to about 99%, such as about 60% to about 98%, or about 70% to about 95% by weight, relative to the total weight of the composition. In at least one embodiment, the solvent consists of or consists essentially of water, with the solvent comprising less than about 10%, less than about 5%, or less than about 3% by weight, relative to the total weight of the composition, of non-aqueous solvents.

Optionally, the compositions useful in the methods of styling, straightening, and/or reducing frizz of the hair may further comprise at least one silicone compound and/or at least one oil. If present, the at least one silicone compound may, in various embodiments, be chosen from dimethicone, dimethicone copolymers, amino functional silicones, or mixtures thereof, and may, for example, be present in an amount ranging from about 0.1% to about 10% by weight, relative to the total weight of the composition, such as from about 0.5% to about 6%, or about 1% to about 5%. If present, the at least one oil may, for example, be chosen from hydrocarbon-based oils of plant origin and/or vegetable origin, or glyceride triesters, and may, in various embodiments, be present in an amount ranging from about 0.1% to about 10% by weight, relative to the total weight of the composition, such as from about 0.5% to about 5%, or about 1% to about 3%.

Optionally, in order to style, straighten, and/or reduce frizz of the hair, the compositions are left on the hair for a period of a few hours to a few days, for example until the hair is washed. In various embodiments, the compositions and/or methods do not include the use of a reducing agent, and/or do not include a step of heating the hair.

The compositions are not mascara compositions, and are not intended to be applied to the eyelashes or eyebrows. Similarly, the compositions are not make-up compositions, and are not intended to be applied to the skin.

### BRIEF DESCRIPTION OF THE FIGURES

The accompanying figures, which are incorporated herein and constitute part of this specification, illustrate exemplary embodiments of the disclosure, and, together with the general description given above and the description provided herein, serve to demonstrate features of the disclosure.

FIG. 1 is a photograph showing the visual differences between inventive composition 1E (left side; smooth and homogenous) and comparative composition C4 (right side; not smooth or homogenous).

FIG. 2 is a photograph showing side and back views of a head of mannequin hair treated with either composition 1C (left half-head) or C1 (right half-head) after the compositions are applied and while the hair is wet (TO), once the hair is dried (T1), and after the hair has been touched (T2).

FIG. 3 is a photograph showing side and back views of a head of mannequin hair treated with either composition 1D (right half-head) or C2 (left half-head) after the compositions are applied and while the hair is wet (TO), once the hair is dried (T1), and after the hair has been touched (T2).

FIG. 4 is a photograph showing side and back views of a head of mannequin hair treated with either composition 1A (right half-head) or C3 (left half-head) after the compositions are applied and while the hair is wet (TO), once the hair is dried (T1), and after the hair has been touched (T2).

It is to be understood that the foregoing and following descriptions are exemplary and explanatory only, and are not intended to be restrictive of any subject matter claimed.

### DETAILED DESCRIPTION

The disclosure relates to compositions for styling hair, for example compositions for straightening wavy or curly hair and/or reducing frizz of the hair, as well as to methods of using the compositions.

### I. Compositions

In various embodiments, compositions according to the disclosure comprise (a) at least one polyphenol; (b) at least one nonionic polymer; (c) at least one cationic surfactant; (d) at least one emulsifier; and (e) at least one solvent. Optionally, the compositions may further comprise (f) at least one silicone compound; and/or (g) at least one oil. The compositions are optionally leave-in compositions, and may, in various embodiments, be in the form of lotion or cream emulsions.

Compositions according to the disclosure may provide straightening effects to the hair, e.g. temporary straightening effects, and in at least some embodiments may provide one or more additional benefits such as softness, smoothness, shine, and/or frizz control to the hair.

### Polyphenols

Compositions according to the disclosure comprise at least one polyphenol. Polyphenols are phenols with more than one phenolic —OH group that have the ability to act as “donor molecules” by donating their alcoholic hydrogen or accepting delocalized electrons. Useful polyphenols include both flavonoids and non-flavonoids.

Exemplary and non-limiting flavonoid compounds that can be used include, for example, flavanols, such as catechin, fisetin, kaempferol, myricetin, quercetin, rutin, proanthocyanidins, pyroanthocyanidins, theaflavins, or thearubigins (or thearubrins); dihydroflavonols, such as astilbin, dihydroquercetin, or silibinin; flavanones, such as hesperidin, neohesperidin, hesperetin, naringenin, naringin, or poncirin; flavones, such as apigenin, baicalin, diosmin, or rhoifolin; tannins, such as ellagitannins, tannic acid, gallic acid, or ellagic acid; isoflavonoids, such as biochanin A, Daidzein, or Genistein; fulvic acid, and neoflavanoids, as well as combinations thereof.

Exemplary and non-limiting non-flavonoid compounds that can be used include, for example, stibenoids such as astringin, resveratrol, or rhaponticin, as well as combinations thereof.

Other polyphenols that can be used include hydroxycinnamic acids, for example, chlorogenic acid, verbascoside; phenolic aldehydes; phenylpropenes; coumarins, coumestans, or tyrosols, as well as combinations thereof. In one embodiment, the polyphenols may be plant-based and/or organic.

In certain exemplary embodiments, polyphenols useful according to the disclosure may be chosen from tannic acid, resveratrol, catechin, ellagic acid, resorcinol, gallic acid, humic acid, chlorogenic acid, quercetin, anthocyanin, chebulinic acid, or mixtures thereof. In certain embodiments, the polyphenols are chosen from polyphenols that do not impart color to the hair. In an exemplary and non-limiting embodiment, the polyphenol comprises tannic acid.

In various embodiments, the at least one polyphenol is present in the compositions in an amount of about 0.01% to about 10% by weight, such as about 0.1% to about 8%, about 0.5% to about 5%, or about 1% to about 3% by weight, relative to the total weight of the composition. By way of non-limiting example, the at least one polyphenol is present in the composition with a total amount of polyphenols ranging from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, from about 0.75% to about 3%, from about 1% to about 10%, from about 1% to about 9%, from about 1% to about 8%, from about 1% to about 7%, from about 1% to about 6%, from about 1% to about 5%, from about 1% to about 4%, or from about 1% to about 3% by weight, relative to the total weight of the composition, including all ranges and subranges thereof.

In some embodiments, the at least one polyphenol comprises, consists essentially of, or consists of tannic acid, where the total amount of tannic acid in the composition ranges from about 0.1% to about 5%, such as about 0.1% to about 4%, about 0.1% to about 3%, about 1% to about 5%, about 1% to about 4%, or about 1% to about 3% by weight, relative to the total weight of the composition, including all ranges and subranges thereof.

### Nonionic Polymers

Compositions according to the disclosure comprise at least one nonionic polymer, which may optionally be a nonionic polysaccharide polymer. Without being bound by theory, the nonionic polymer is believed to aid in straightening the hair in the absence of reducing agents and/or heat, when used in combination with other components of the compositions according to the disclosure. Optionally, the compositions may be free or substantially free of anionic polymers.

In various embodiments, the at least one nonionic polymer may be a nonionic polysaccharide polymer chosen from, by way of non-limiting example only, nonionic C1-C3 alkyl guars, nonionic C1-C3 hydroxyalkyl guars, glucans, modified or non-modified starches, dextrans, celluloses and derivatives thereof, or mixtures thereof. For example, the nonionic polysaccharide polymer may be chosen from guar gum, hydroxypropyl guar, methylcelluloses, hydroxyalkylcelluloses such as hydroxyethylcellulose, caesalpinia spinosa gum, sclerotium gum, locust bean gum, pullulan, or mixtures thereof. In at least one exemplary embodiment, the at least one nonionic polysaccharide polymer is a glucan, for example sclerotium gum.

In various embodiments, the at least one nonionic polysaccharide polymer may be present in an amount ranging from about 0.01% to about 5%, such as from about 0.01% to about 4%, about 0.01% to about 3%, about 0.01% to about 2%, about 0.01% to about 1%, about 0.01% to about 0.8%, about 0.05% to about 5%, about 0.05% to about 4%, about 0.05% to about 3%, about 0.05% to about 2%, about 0.05% to about 1%, about 0.05% to about 0.8%, about 0.1% to about 5%, about 0.1% to about 4%, about 0.1% to about 3%, about 0.1% to about 2%, about 0.1% to about 1%, about 0.1% to about 0.9%, about 0.1% to about 0.8%, about 0.1% to about 0.7%, or about 0.1% to about 0.6% by weight, including all ranges and sub-ranges there between, based on the total weight of the composition.

In various embodiments, the compositions comprise less than about 0.5%, such as less than about 0.4%, less than about 0.3%, less than about 0.2%, less than about 0.1%, less than about 0.075%, less than about 0.05%, less than about 0.025%, less than about 0.01%, less than about 0.005%, or less than about 0.001% of anionic polymers by weight, relative to the total weight of the composition.

In one exemplary embodiment, the at least one nonionic polysaccharide polymer comprises, consists essentially of, or consists of sclerotium gum, where the total amount of sclerotium gum in the composition ranges from about 0.1% to about 5%, about 0.1% to about 4%, about 0.1% to about 3%, about 0.1% to about 2%, about 0.1% to about 1%, about 0.1% to about 0.9%, about 0.1% to about 0.8%, about 0.1% to about 0.7%, or about 0.1% to about 0.6% by weight, including all ranges and sub-ranges there between, based on the total weight of the composition.

In some embodiments, the at least one nonionic polymer is chosen from nonionic polymers that are not polysaccharides, but which nevertheless provide hair styling, straightening, and/or frizz-reduction benefits to the hair styling compositions according to the disclosure. For example, the nonionic polymer may be chosen from homopolymers and copolymers of ethylene oxide, polyvinyl alcohols, homopolymers and copolymers of vinylpyrrolidone, homopolymers and copolymers of vinylcaprolactam, homopolymers and copolymers of polyvinyl methyl ether, neutral acrylic homopolymers and copolymers, or mixtures thereof. In certain embodiments, the at least one nonionic polymer may be chosen from polyvinylpyrrolidones, polyvinylcaprolactams, polyvinylpyrrolidone/vinyl acetate copolymers, polyvinylpyrrolidone/vinyl acetate/vinyl propionate terpolymers, vinyl caprolactam/VP/di-methylaminoethyl methacrylate copolymers, or mixtures thereof.

Nonionic polymers that are not polysaccharides may, in various embodiments, advantageously be chosen when the pH of the hair styling composition is less than about 6, such as less than about 5.8, less than about 5.5, less than about 5.3, or less than about 5. In further embodiments, nonionic polymers that are not polysaccharides may advantageously be chosen when the hair styling composition comprises water in an amount of at least about 50%, such as at least about 55%, at least about 60%, at least about 65%, or at least about 70% by weight, relative to the total weight of the hair styling composition. In one exemplary and non-limiting embodiment, the at least one nonionic polymer may be chosen from polyvinypyrrolidone, for example low molecular weight polyvinylpyrrolidone.

In various embodiments, the at least one nonionic polymer that is not a polysaccharide may be present in the hair styling compositions in an amount sufficient to provide hair styling, straightening, and/or frizz-reduction benefits to the compositions according to the disclosure. For example, the nonionic polymer that is not a polysaccharide may be present in an amount ranging from about 0.01% to about 5%, such as from about 0.01% to about 4%, about 0.01% to about 3%, about 0.01% to about 2%, about 0.01% to about 1%, about 0.01% to about 0.8%, about 0.05% to about 5%, about 0.05% to about 4%, about 0.05% to about 3%, about 0.05% to about 2%, about 0.05% to about 1%, about 0.05% to about 0.8%, about 0.1% to about 5%, about 0.1% to about 4%, about 0.1% to about 3%, about 0.1% to about 2%, about 0.1% to about 1%, about 0.1% to about 0.9%, about 0.1% to about 0.8%, about 0.1% to about 0.7%, or about 0.1% to about 0.6% by weight, including all ranges and sub-ranges there between, based on the total weight of the hair styling composition.

Mixtures of nonionic polysaccharide polymers and nonionic polymers that are not polysaccharides may also be chosen.

### Cationic Surfactants

Compositions according to the disclosure comprise at least one cationic surfactant. The term “cationic surfactant” means a surfactant comprising, as ionic or ionizable groups, only cationic groups. In certain embodiments, the cationic surfactants are plant-based and/or organic. Optionally, in at least some embodiments, the compositions may be free or substantially free of nonionic surfactants.

In some embodiments, a cationic surfactant may be chosen from amidoamine compounds (or amidoamines). Examples of amidoamines that are useful in the compositions of the instant disclosure include, but are not limited to the following: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicamidopropyl dimethylamine, olivamidopropyl dimethylamine, palmitamidopropyl dimethylamine, stearamidoethyldiethylamine, and mixtures thereof.

In some further embodiments, a cationic surfactant can be chosen from monoalkyl quaternary amines, dialkyl quaternary amines, or polyquaternium compounds or salts thereof.

For example, a cationic surfactant may be chosen from Polyquaterium-10 (also called quaternized polyhydroxyethyl cellulose), cetrimonium chloride, behentrimonium chloride, behentrimonium methosulfate, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, Quaternium-91, Polyquaternium-37 (e.g., under the SALCARE tradename), Quaternium-22, Quaternium-87, Polyquaternium-4, Polyquaternium-6, Polyquaternium-11, Polyquaternium-44, Polyquaternium-67, amodimethicone, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, aminopropyl dimethicone, Quaterium-8, dilinoleamidopropyl dimethylamine dimethicone PEG-7 phosphate, or mixtures thereof.

In some embodiments, a suitable cationic surfactant may be chosen from polyquaternium 4, polyquaternium 6, polyquaternium 7, polyquaternium 10, polyquaternium 11, polyquaternium 16, polyquaternium 22, polyquaternium 28, polyquaternium-32, polyquaternium-46, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-63, polyquaternium-64, polyquaternium-65, polyquaternium-66, polyquaternium-67, polyquaternium-70, polyquaternium-73, polyquaternium-74, polyquaternium-75, polyquaternium-76, polyquaternium-77, polyquaternium-78, polyquaternium-79, polyquaternium-80, polyquaternium-81, polyquaternium-82, polyquaternium-84, polyquaternium-85, polyquaternium-86, polyquaternium-87, polyquaternium-90, polyquaternium-91, polyquaternium-92, polyquaternium-94, or guar hydroxypropyltrimonium chloride, and mixtures thereof. In an embodiment, the cationic surfactant is chosen from Polyquaternium-67, Polyquaternium-10, Polyquaternium-37, or mixtures thereof. Polyquaternium-37 may be commercially available from BASF under the tradename of SALCARE SC 96 (comprising Polyquaternium-37 (and) Propylene Glycol Dicaprylate/Dicaprate (and) PPG-1 Trideceth-6).

In some embodiments, the cationic surfactant in the compositions comprises behentrimonium chloride. In some further embodiments, the at least one cationic surfactant is chosen from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyl-diethylamine, behenamidoethyldimethylamine, arachidamidopropyldimethylamine, arachidamido-propyidiethylamine, arachidamidoethyldiethylamine, arachidamidoethyl-dimethylamine, or a combination of any two or more thereof.

Non-limiting examples of cationic surfactants may also include behenalkonium chloride, benzethonium chloride, cetylpyridinium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine hydrochloride, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, hexadecyltrimethyl ammonium bromide, or mixtures thereof.

In some embodiments, the at least one cationic surfactant is chosen from polyoxyalkylenated primary, secondary, or tertiary fatty amine salts, quaternary ammonium salts, or mixtures thereof. In some cases it is useful to use salts such as chloride salts of the quaternary ammonium compounds. The fatty amines may comprise at least one C8-C30 hydrocarbon-based chain.

In some embodiments, suitable quaternary ammonium salts may be those of formula (I):

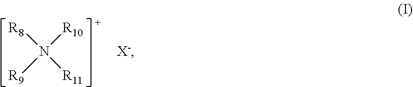

wherein:

groups R8 to R11 are independently chosen from linear or branched aliphatic groups containing from 1 to 30 carbon atoms, or an aromatic group such as aryl or alkylaryl, optionally wherein at least one of the groups R8 to R11 includes from 8 to 30 carbon atoms, such as from 12 to 24 carbon atoms, it being possible for the linear or branched aliphatic groups to include heteroatoms such as, for example, oxygen, nitrogen, and/or sulfur, these heteroatoms not being adjacent, and halogens; and

X⁻ is an anion chosen from the group consisting of halides such as bromides, chlorides, iodides, fluorides, phosphates, acetates, lactates, (C1-C4)alkyl sulfates, (C1-C4)alkyl sulfonates or (C1-C4)alkylaryl sulfonates; C1-C30 alkyl, C1-C30 alkoxy, (C2-C6)polyoxyalkylene, C1-C30 alkylamide, (C12-C22)alkyl-(C2C6)alkylamido, (C12-C22)alkyl acetate, and C1-C30 hydroxyalkyl groups.

Mention may be made as exemplary embodiments of formula (I) of tetraalkylammonium halides, such as chlorides, for example dialkyldimethylammonium or alkyltrimethylammonium chlorides in which the alkyl group comprises from 12 to 22 carbon atoms, such as from 14 to 20 carbon atoms. By way of example, behenyltrimethylammonium chloride (behentrimonium chloride), distearyl-dimethylammonium chloride, cetyltrimethylammonium chloride (cetrimonium chloride), or benzyldimethylstearylammonium chloride may be chosen.

In some embodiments, compounds of formula (I) may include palmitylamidopropyltrimethylammonium or stearamidopropyldimethyl-(myristyl acetate)-ammonium halides, such as chlorides, for example the product sold under the name CERAPHYL® 70 by the company Van Dyk.

In certain embodiments, cationic surfactant of formula (I) are preferably chosen from alkyltrimethylammonium halides whose alkyl group includes from 12 to 22 carbon atoms, such as from 14 to 20 carbon atoms. For example, alkyltrimethylammonium chlorides, such as behenyltrimethylammonium chloride and cetyltrimethylammonium chloride, may be particularly useful.

In further embodiments, quaternary ammonium salts of imidazoline of formula (II) may be chosen:

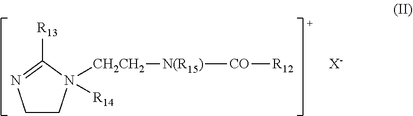

wherein:

R12 represents an alkenyl or alkyl group comprising from 8 to 30 carbon atoms, for example derived from tallow fatty acids,

R13 represents a hydrogen atom, a C1-C4 alkyl group or an alkenyl or alkyl group comprising from 8 to 30 carbon atoms,

R14 represents a C1-C4 alkyl group,

R15 represents a hydrogen atom or a C1-C4 alkyl group, and

X⁻ is an anion chosen from the group consisting of halides, phosphates, acetates, lactates, (C1-C4)alkyl sulfates, and (C1-C4)alkyl- or (C1-C4)alkylarylsulfonates.

In one exemplary embodiment of formula (II), R12 and R13 represent a mixture of alkenyl or alkyl groups containing from 12 to 21 carbon atoms, derived for example from tallow fatty acids; R14 represents a methyl group; and R15 represents a hydrogen atom. Such a product may be sold, for example, under the name REWOQUAT® W 75 by the company Evonik.

In yet further embodiments, di- or triquaternary ammonium salts of formula (III) may be chosen:

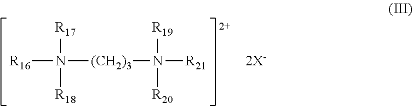

wherein:

R16 represents an alkyl group comprising from 16 to 30 carbon atoms, which is optionally hydroxylated and/or optionally interrupted with one or more oxygen atoms,

R17 represents hydrogen, an alkyl group comprising from 1 to 4 carbon atoms, or a group —(CH₂)₃—N⁺(R16a)(R17a)(R18a),

R16a, R17a and R18a, which may be identical or different, represent hydrogen or an alkyl group comprising from 1 to 4 carbon atoms,

R18, R19, R20, and R21, which may be identical or different, represent hydrogen or an alkyl group comprising from 1 to 4 carbon atoms, and

X⁻ is an anion chosen from the group consisting of halides, acetates, phosphates, nitrates, (C1-C4)alkyl sulfates, and (C1-C4)alkyl- and (C1-C4)alkylarylsulfonates, for example methyl sulfate or ethyl sulfate.

Such compounds are, for example, FINQUAT® CT-P (Quaternium 89) and FINQUAT® CT (Quaternium 75), sold by the company Finetex.

In still further embodiments, quaternary ammonium salts containing one or more ester functions, such as those of formula (IV) may be chosen:

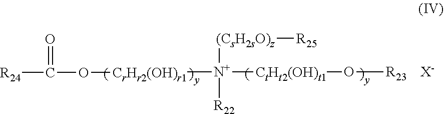

wherein:

R22 is chosen from C1-C6 alkyl groups and C1-C6 hydroxyalkyl or dihydroxyalkyl groups,

R23 is chosen from the group R26-C(═O)—; linear or branched, saturated or unsaturated C1-C22 hydrocarbon-based groups; and a hydrogen atom,

R25 is chosen from the group R28-C(═O)—; linear or branched, saturated or unsaturated C1-C6 hydrocarbon-based groups; and a hydrogen atom,

R24, R26, and R28, which may be identical or different, are chosen from saturated or unsaturated, linear or branched C7-C21 hydrocarbon-based groups,

r, s, and t, which may be identical or different, are integers ranging from 2 to 6,

r1 and t1, which may be identical or different, are equal to 0 or 1,

y is an integer ranging from 1 to 10,

x and z, which may be identical or different, are integers ranging from 0 to 10, and

X⁻ is an anion;
it being understood that r2+r1=2r and t1+t2=2t, and that the sum x+y+z ranges from 1 to 15, with the proviso that when x=0 then R23 is chosen from C1-C22 hydrocarbon-based groups, and that when z=0 then R25 denotes a linear or branched, saturated or unsaturated C1-C6 hydrocarbon-based group.

In exemplary embodiments of formula (IV), the alkyl groups R22 may be linear or branched, and are preferably linear. Preferably, R22 denotes a methyl, ethyl, hydroxyethyl or dihydroxypropyl group, and more particularly a methyl or ethyl group. Advantageously, the sum x+y+z ranges from 1 to 10. When R23 is a C1-C22 hydrocarbon-based groups, it may preferably comprise either from 12 to 22 carbon atoms or from 1 to 3 carbon atoms. Advantageously, R24, R26 and R28, which may be identical or different, are chosen from linear or branched, saturated or unsaturated C11-C21 hydrocarbon-based groups, and more particularly from linear or branched C11-C21 alkyl and alkenyl groups. Optionally, x and z, which may be identical or different, are equal to 0 or 1. Optionally, y is equal to 1. Optionally, r, s, and t, which may be identical or different, are equal to 2 or 3, and optionally are equal to 2.

The anion X⁻ is optionally a halide, e.g. chloride, bromide, or iodide, a (C1-C4)alkyl sulfate, a (C1-C4)alkylsulfonate, or a (C1-C4)alkylarylsulfonate, a methanesulfonate, a phosphate, a nitrate, a tosylate, an anion derived from an organic acid such as an acetate or a lactate, or any other anion that is compatible with the ammonium bearing an ester function. In some embodiments, the anion X⁻is a chloride, a methyl sulfate, or an ethyl sulfate.

For example, the ammonium salts of formula (IV), in which R22 is a methyl or ethyl group, x and y are equal to 1, z is equal to 0 or 1, r, s and t are equal to 2, R23 is chosen from the group R26-C(═O)—, methyl, ethyl, or C14-C22 hydrocarbon-based groups, and a hydrogen atom; R25 is chosen from the group R28-C(═O)—, and a hydrogen atom; R24, R26 and R28, which may be identical or different, are chosen from linear or branched, saturated or unsaturated C13-C17 hydrocarbon-based groups, and preferably from linear or branched, saturated or unsaturated C13-C17 alkyl and alkenyl groups, may be chosen. In one embodiment, the hydrocarbon-based groups are linear.

Among the compounds having formula (IV), mention may be made of salts, especially the chloride or methyl sulfate salts, of diacyloxyethyldimethylammonium, diacyloxyethylhydroxyethylmethylammonium, monoacyloxyethyldihydroxyethylmethyl-ammonium, triacyloxyethylmethylammonium or monoacyloxyethylhydroxyethyldimethyl-ammonium, and mixtures thereof. The acyl groups preferably contain 14 to 18 carbon atoms and are obtained more particularly from a plant oil such as palm oil or sunflower oil. When the compound contains several acyl groups, these groups may be identical or different.

These cationic surfactants may be obtained, for example, by direct esterification of triethanolamine, triisopropanolamine, alkyldiethanolamine or alkyldiisopropanolamine, which are optionally oxyalkylenated, with fatty acids or with fatty acid mixtures, such as those of plant or animal origin, or by transesterification of the methyl esters thereof. This esterification may be followed by a quaternization by means of an alkylating agent, such as an alkyl halide, for example, methyl or ethyl halide, a dialkyl sulfate, for example, dimethyl or diethyl sulfate, methyl methanesulfonate, methyl para-toluenesulfonate, glycol chlorohydrin, or glycerol chlorohydrin. Such compounds are sold, for example, under the names DEHYQUART® by the company Henkel, STEPANQUAT® by the company Stepan, NOXAMIUM® by the company CECA or REWOQUAT® WE 18 by the company Evonik.

In some embodiments, the at least one cationic surfactant may be chosen from, for example, a mixture of quaternary ammonium monoester, diester, and triester salts with a weight majority of diester salts. In some embodiments, the at least one cationic surfactant may be chosen from the ammonium salts containing at least one ester functional group that are described in patents U.S. Pat. Nos. 4,874,554 and 4,137,180, which are incorporated herein for their entireties. In some embodiments, the at least one cationic surfactant may comprise behenoylhydroxypropyl-trimethylammonium chloride, for example, sold by the company Kao under the name QUARTAMIN® BTC 131.

Suitable cationic surfactants may further include those of formula (V):

R4-A-R5-B (V),

wherein R4 is a saturated or unsaturated, straight or branched alkyl chain with 8 to 24 carbon atoms, R5 is a straight or branched alkyl chain with 1 to 4 carbon atoms, A is selected from:

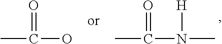

and B is selected from compounds of formula (VI):

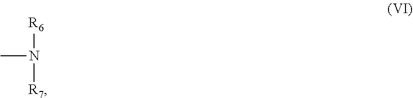

wherein R6 and R7, which may be the same or different, are chosen from H, an alkyl chain with 1 to 4 carbon atoms, hydroxyl alkyl chain with 1 to 4 carbon atoms, or di hydroxyl alkyl chain with 2 to 4 carbon atoms; or compounds of formula (VII):

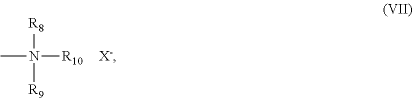

wherein R8 and R9, which can be the same or different, are chosen from an alkyl chain with 1 to 4 carbon atoms, a hydroxyl alkyl chain with 1 to 4 carbon atoms, or a di-hydroxyl alkyl chain with 2 to 4 carbon atoms; wherein R10 is chosen from an alkyl chain with 1 to 4 carbon atoms, a hydroxyl alkyl chain with 1 to 4 carbon atoms, or a di-hydroxyl alkyl chain with 2 to 4 carbon atoms.

In some instances, in formula (V), R4 is saturated or unsaturated, straight or branched alkyl chain with 10 to 24 carbon atoms, more preferably 12 to 22 carbon atoms, R5 is straight or branched alkyl group with 1 to 4 carbon atoms, and A, B, R6 to R10 are same as above.

Non-limiting suitable examples include stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl buylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropyl amine, palmitoylpropyl methylamine, palmitoylpropyl diethylamine, palmitoylpropyl dibutylamine, palmitoylpropyl buylamine, palmitoylpropyl dipropylamine, palmitoylpropyl propylamine, palmitoylpropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

In some embodiments, a cationic surfactant may be chosen from cationizable surfactants such as fatty alkylamines, preferably, fatty dialkylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and mixtures thereof.

Fatty dialkylamines include fatty amidoamine compounds, their salts, and mixtures thereof. Non-limiting examples include oleamidopropyl dimethylamine, linoleamidopropyl dimethylamine, isostearamidopropyl dimethylamine, stearamidopropyl dimethylamine, oleyl hydroxyethyl imidazoline, stearamidopropyldimethylamine, behenamidopropyldimethylamine, behenamidopropyldiethylamine, behenamidoethyl-diethylamine, behenamidoethyldimethylamine, arachidamidopropyldimethylamine, arachidamido-propyidiethylamine, arachidamidoethyldiethylamine, arachidamidoethyl-dimethylamine, brassicamidopropyldimethylamine, lauramidopropyl dimethylamine, myristamidopropyl dimethylamine, dilinoleamidopropyl dimethylamine, palmitamidopropyl dimethylamine, or mixtures thereof.

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. In some embodiment, a carboxylic acid may be chosen from lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, or mixtures thereof. In some embodiment, lactic acid, tartaric acid or mixtures thereof are used, especially in combination with fatty dimethylamines such as, for example, stearamidopropyl dimethylamine.

Compositions according to the disclosure may be formulated such that two or more cationic surfactants are associated with the same or different balancing anionic ions. For example, at least one of the two or more cationic surfactants may have a chloride ion and/or a sulfate ion. In some embodiments, cationic surfactants are chosen from cetrimonium chloride, behentrimonium methosulfate, behentrimonium chloride, or combinations thereof. In some further embodiments, the cationic surfactants comprise behentrimonium chloride and one or both of behentrimonium methosulfate and cetrimonium chloride.

In various embodiments, the total amount of the at least one cationic surfactant may range up to about 15%, based on the total weight of the composition, including all ranges and subranges therebetween. For instance, the total amount of the at least one cationic surfactant may range from about 0.01% to about 10%, from about 0.1% to about 8%, from about 0.5% to about 5%, or from about 0.75% to about 3% by weight, relative to the total weight of the composition. In certain embodiments, the at least one cationic surfactant may be present in an amount ranging from about 0.01% to about 5%, about 0.01% to about 4%, about 0.01% to about 3%, about 0.01% to about 2%, about 0.01% to about 1%, about 0.05% to about 5%, about 0.05% to about 4%, about 0.05% to about 3%, about 0.05% to about 2%, about 0.05% to about 1%, about 0.1% to about 5%, about 0.1% to about 4%, about 0.1% to about 3%, about 0.1% to about 2%, about 0.1% to about 1%, about 0.5% to about 5%, about 0.5% to about 4%, about 0.5% to about 3%, about 0.5% to about 2%, or about 0.5% to about 1% by weight, based on the total weight of the composition, including all ranges and subranges thereof.

### Emulsifiers

Compositions according to the disclosure comprise at least one emulsifier. In various embodiments, the one or more emulsifiers may be chosen from (i) fatty alcohols, (ii) esters, or (iii) combinations thereof.

### Fatty Alcohols

In some embodiments, compositions according to the disclosure comprise at least one emulsifier chosen from fatty alcohols or derivatives thereof. In certain embodiments, the compositions comprise at least two fatty alcohols.

As used herein, “fatty alcohol” refers to any alcohol with a carbon chain of C5 or greater, such as, for example, C8 or greater, C10 or greater, and C12 or greater. Suitable fatty alcohols according to the disclosure may include, but are not limited to, alkoxylated or non-alkoxylated, saturated or unsaturated, linear or branched, fatty alcohols, for example with from 6 to 30 carbon atoms, such as from 8 to 30 carbon atoms, from 8 to 22 carbon atoms, from 12 to 22 carbon atoms, or from 12 to 18 carbon atoms, including all ranges and subranges therebetween. The fatty alcohols may be liquid or solid. Non-limiting examples of the fatty alcohols and derivatives thereof are found in International Cosmetic Ingredient Dictionary, Sixteenth Edition, 2016, which is incorporated by reference herein.

As used herein, “alkoxylated fatty alcohol” refers to any fatty alcohol with a carbon chain of C5 or greater, as defined above, further comprising at least one alkoxy group. For example, the at least one alkoxylated fatty alcohol may have a carbon chain of C8 or greater, C10 or greater, and C12 or greater. Further, for example, the at least one alkoxylated fatty alcohol may be chosen from alkoxylated polymers (including co-, ter- and homo-polymers) derived from alcohols such as glycerol (e.g., polyglyceryl derived from four glycerol molecules). The at least one alkoxy group of the at least one alkoxylated fatty alcohol may, for example, be derived from an alkoxylation reaction carried out with alkylene oxide. Non-limiting examples of at least one alkoxylated fatty alcohol include any fatty alcohol comprising at least one polyethylene glycol ether and any fatty alcohol comprising at least one polypropylene glycol ether.

In some embodiments, the compositions according to the disclosure comprise a liquid fatty alcohol. In some embodiments, the compositions according to the disclosure comprise a solid fatty alcohol. In further embodiments, the compositions according to the disclosure comprise combinations of at least one liquid fatty alcohol and at least one solid fatty alcohol.

According to some embodiments, liquid fatty alcohols that can be used may contain C10-C34, and may have branched carbon chains and/or have branched and/or unsaturated (C=C double bond), and contain from 12 to 40 carbon atoms.

The liquid fatty alcohols may, in various embodiments, be represented by the formula R—OH, wherein R denotes a C12-C24 branched alkyl group or an alkenyl group (comprising at least one C12-C24 double bond C═C), R being optionally substituted by one or more hydroxy groups. In some embodiments, a liquid fatty alcohol is a branched saturated alcohol. In at least certain embodiments, R does not contain a hydroxyl group. Non-limiting examples of liquid fatty alcohols may include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, and 2-tetradecyl-1-cetanol. In some particular embodiments, the liquid fatty alcohol is 2-octyl-1-dodecanol.

According to further embodiments, solid fatty alcohols that can be used may include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm. The solid fatty alcohols may be soluble, under the same temperature and pressure conditions, in at least one organic solvent (for example ethanol, chloroform, benzene or liquid petroleum jelly) to at least 1% by weight.

The solid fatty alcohols may, in various embodiments, be represented by the formula R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

Non-limiting examples of solid fatty alcohols include lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol); or mixtures thereof. In some embodiments, the compositions disclosed herein comprise at least one solid fatty alcohol chosen from cetyl alcohol, stearyl alcohol, behenyl alcohol and mixtures thereof such as cetylstearyl or cetearyl alcohol.

The at least one emulsifier may also be chosen from fatty alcohol derivatives, as well as mixtures of fatty alcohols, mixtures of fatty alcohol derivatives, and mixtures of fatty alcohols and fatty alcohol derivatives.

In some embodiments, compositions according to the disclosure comprise a fatty alcohol that is hydrogenated (for example, stearyl, lauryl, cetyl, cetearyl). In some embodiments, the fatty alcohol contains one or more double bonds (for example, oleyl). In some embodiments, an emulsifier comprised in the compositions is chosen from decyl alcohol, undecyl alcohol, dodecyl alcohol, myristyl alcohol, lauryl alcohol, cetyl alcohol, stearyl alcohol, cetearyl alcohol (a combination of cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, or a combination thereof. In some embodiments, an emulsifier comprised in the compositions may be chosen from myristyl alcohol, lauryl alcohol, cetyl alcohol, stearyl alcohol, cetearyl alcohol, isostearyl alcohol, oleyl alcohol, isotridecyl alcohol, or a combination thereof.

Thus, without intending to be limiting, the at least one emulsifier may be chosen from C9-C11 alcohols, C12-C13 alcohols, C12-C15 alcohols, C12-C16 alcohols, C14-C15 alcohols, C12-C22 alcohols, arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, 2-octyldodecanol, isostearyl alcohol, 2-hexyldecanol, 2-heptyldecanol, 2-octyldecanol, caproic alcohol (1-hexanol), enanthic alcohol (1-heptanol), caprylic alcohol (1-octanol), pelargonic alcohol (1-nonanol), capric alcohol (1-decanol), lauryl alcohol (1-dodecanol), or mixtures thereof.

In at least one embodiment, the at least one fatty alcohol comprises, consists essentially of, or consists of cetyl alcohol. In at least one embodiment, the at least one fatty alcohol comprises, consists essentially of, or consists of cetearyl alcohol. In at least one embodiment, the at least one fatty alcohol comprises, consists essentially of, or consists of cetyl alcohol and cetearyl alcohol.

### Glyceryl Esters (Glycerol Esters)

In some embodiments, compositions according to the disclosure comprise at least one emulsifier chosen from one glyceryl (or glycerol) esters, or derivatives thereof.

The glyceryl esters may, in some embodiments, have a carbon chain of 8 to 24 carbons. Non-limiting examples of glyceryl esters include bis-diglyceryl polyacyladipate-2, glyceryl behenate, glyceryl caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate, glyceryl dioleate, glyceryl distearate, glyceryl laurate, or mixtures thereof.

### Non-Glyceryl Esters

In some embodiments, compositions according to the disclosure may also include an ester other than glyceryl esters (non-glyceryl esters). In some cases, the ester other than glyceryl ester is chosen from isopropyl esters, cetyl esters, fatty esters, or a mixture thereof. Non-limiting examples of isopropyl esters include isopropyl myristate, isopropyl laurate, isopropyl oleate, isopropyl palmitate, and isopropyl stearate. In some embodiments, the ester that is not a glyceryl ester may be chosen from isopropyl myristate, cetyl esters, isopropyl palmitate, or mixtures thereof.

According to various embodiments, the total amount of the at least one emulsifier chosen from fatty alcohols, esters, and/or derivatives thereof, or the amount of each of the individual fatty alcohols, esters, and/or derivatives thereof, in the composition may range up to about 15%, such from about 0.01% up to about 15% by weight, based on the total weight of the composition. By way of non-limiting example, any of the at least one emulsifier chosen from fatty alcohols, esters, or combinations thereof, may be present in the composition in an amount ranging from about 0.01% to about 10%, about 0.01% to about 9%, about 0.01% to about 8%, about 0.01% to about 7%, about 0.01% to about 6%, about 0.1% to about 10%, about 0.1% to about 9%, about 0.1% to about 8%, about 0.1% to about 7%, or about 0.1% to about 6% by weight, based on the weight of the composition, including ranges and subranges thereof.

In certain embodiments where more than one emulsifier is present, each emulsifier is present in an amount ranging from about 0.5% to about 8%, such as about 1% to about 7% by weight, based on the weight of the composition. In other embodiments where more than one emulsifier is present, the total amount of emulsifiers ranges from about 0.5% to about 8%, such as about 1% to about 7% by weight, based on the weight of the composition.

In some embodiments, the composition comprises at least one fatty alcohol and at least one ester. Optionally, the composition comprises at least one fatty alcohol and at least one non-glyceryl ester. In one embodiment, the at least one emulsifier comprises, consists essentially of, or consists of fatty alcohols and esters, in particular non-glyceryl esters. By way of example, the composition may comprise at least one fatty alcohol chosen from cetearyl alcohol and at least one ester chosen from cetyl esters. In certain embodiments where the composition comprises at least one fatty alcohol and at least one ester, the at least one fatty alcohol may be present in the composition in an amount ranging from about 0.1% to about 10%, such as from about 0.1% to about 9%, about 0.1% to about 8%, about 0.1% to about 7%, about 0.1% to about 6%, about 1% to about 10%, about 1% to about 9%, about 1% to about 8%, about 1% to about 7%, or about 1% to about 6% by weight, based on the weight of the composition, including ranges and subranges thereof, and the at least one ester may be present in an amount ranging from about 0.01% to about 5%, such as from about 0.01% to about 4%, about 0.01% to about 3%, about 0.01% to about 2%, about 0.01% to about 1.5%, about 0.01% to about 1%, about 0.05% to about 5%, about 0.05% to about 4%, about 0.05% to about 3%, about 0.05% to about 2%, about 0.05% to about 1.5%, about 0.05% to about 1%, about 0.1% to about 5%, about 0.1% to about 4%, about 0.1% to about 3%, about 0.1% to about 2%, about 0.1% to about 1.5%, or about 0.1% to about 1% by weight, including all ranges and sub-ranges there between, based on the total weight of the composition.

### Solvent

Compositions according to the disclosure comprise one or more cosmetically acceptable solvents. The solvent(s) may be chosen from water, non-aqueous solvents, or mixtures thereof. The total amount of solvent in the composition may range from about 50% to about 99% by weight, relative to the total weight of the composition, including all ranges and subranges therebetween. For example, in one embodiment, the total amount of solvent may be about 50% to about 98%, about 60% to about 95%, about 70% to about 90%, about 70% to about 85%, or about 75% to about 85% by weight, relative to the total weight of the composition.

In one embodiment, the solvent comprises water. In other embodiments, the composition comprises water and one or more non-aqueous solvents. For example, C₁-C₄ alcohols, organic solvents, polyols, glycols, hydrocarbons/alkanes, glycol ethers, or any mixture thereof. Non-limiting examples of solvents which may be used include alkane polyols such as, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-isopropyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-isopropyl ether, dipropylene glycol monomethyl ether, dipropylene glycol monoethyl ether, dipropylene glycol mono-n-propyl ether, and dipropylene glycol mono-isopropyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and mixtures thereof.

In some exemplary embodiments, the solvent comprises water and at least one additional solvent chosen from linear or branched, saturated or unsaturated, and substituted or unsubstituted polyols, for example C2-C8 polyols or C3-C6 polyols, such as diols and/or triols. Any stereoisomer of the polyols may be used. In various exemplary embodiments, the C2-C8 polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, and mixtures thereof.

In various embodiments, the solvent consists of, or consists essentially of, water. In certain embodiments, the solvent comprises water in an amount ranging from about 50% to about 95%, about 60% to about 90%, about 70% to about 85%, or about 75% to about 80%, by weight of the solvent, with the remainder of the solvent comprising one or more additional solvents. For example, in certain embodiments, the composition comprises about 50% to about 99%, about 60% to about 98%, about 70% to about 95%, or about 75% to about 93% water, by weight of the composition. In other embodiments, the compositions comprise water and at least one non-aqueous solvent, where the total amount of non-aqueous solvent is present in an amount less than about 20%, such as less than about 18%, less than about 16%, less than about 15%, less than about 13%, less than about 12%, less than about 10%, less than 8%, less than 5%, or less than 3% by weight, relative to the total weight of the composition. In some embodiments, the compositions are free or substantially free of non-aqueous solvents.

### Silicones

Compositions according to the disclosure optionally contain at least one silicone compound. In various embodiments, if present, the at least one silicone compound may be chosen from, for example, silicone acrylate polymers, hydroxylmethylpolysiloxane (methicone), amino functional silicones such as amodimethicones, dimethicone copolyols, polydimethylsiloxanes (dimethicones), polydiethylsiloxanes, polydimethyl siloxanes having terminal hydroxyl groups (dimethiconols), or mixtures thereof.

In some embodiments, the compositions comprise amodimethicone. In further embodiments, mixtures of silicone compounds, for example silicone emulsions, may be used. For example, silicone emulsions sold under the name XIAMETER™ by Dow, which are blends of Amodimethicone (and) Trideceth-12 (and) Cetrimonium Chloride or Amodimethicone (and) Trideceth-6 (and) Cetrimonium Chloride may be chosen.

The amount of the at least one silicone compound or emulsion that may be included in various embodiments can vary but, if present, typically ranges from about 0.1% to about 10%, including all subranges therebetween, such as from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 2% to about 10%, from about 2% to about 9%, from about 2% to about 8%, from about 2% to about 7%, from about 2% to about 6%, from about 2% to about 5%, from about 2% to about 4%, or from about 2% to about 3% by weight, relative to the total weight of the composition, including all ranges and subranges thereof.

### Oils

Compositions according to the disclosure optionally comprise at least one oil, which may, in some embodiments, be a low molecular weight oil. The oil may, in certain embodiments, be chosen from oils which are derived from a plant or vegetable, for example, oils from seeds or fruits.

Oils that may be used according to the disclosure include, by way of example, a hydrocarbon-based oils of plant origin and/or vegetable origin. Non-limiting examples of such oils include acai oil, almond oil, aloe vera oil, andiroba oil, annatto oil, avocado oil, babassu oil, borage oil, brazil nut oil, buriti oil, camelina oil, coffee oil, copaiba oil, emu oil, passion fruit oil, almond oil, castor oil, coconut oil, grapeseed oil, jojoba oil, macadamia nut oil, rose hip oil, ajwain oil, angelic root oil, anise oil, aragan oil, asafetida, balsam oil, basil oil, bay oil, bergamot oil, black pepper essential oil, buchu oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, cypress, cypriol, curry leaf, davana oil, dill oil, elecampane, eucalyptus oil, fennel seed oil, fenugreek oil, fir, frankincense oil, galangal, geranium oil, ginger oil, goldenrod, grapefruit oil, grapeseed oil, henna oil, helichrysum, horseradish oil, hyssop, Idaho tansy, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, patchouli oil, perilla oil, pennyroyal oil, peppermint oil, petitgrain, pine oil, plum oil, ravensara, red cedar, roman chamomile, rose oil, rosehip oil, rosemary oil, rosewood oil, sandalwood oil, sassafras oil, savory oil, schisandra oil, spikenard, spruce, star anise oil, tangerine, tarragon oil, tea tree oil, thyme oil, tsuga oil, turmeric, valerian, vetiver oil, western red cedar, wintergreen, yarrow oil, ylang-ylang, and zedoary oil.

In some embodiments, oils that may be used according to the disclosure may include an oil chosen from glyceride triesters, which are generally triesters of fatty acids and of glycerol, the fatty acids of which can have varied chain lengths from C4 to C24, it being possible for these chains to be saturated or unsaturated and linear or branched. Non-limiting examples of these oils include wheat germ oil, sunflower oil, grape seed oil, sesame oil, corn oil, apricot oil, castor oil, shea oil, avocado oil, olive oil, soybean oil, sweet almond oil, palm oil (which contains palmitic acid), rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.

If present, the total amount of the at least one oil that may be included in various embodiments ranges from about 0.01% to about 15%, such as from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, or from about 1% to about 2% by weight, based on the total weight of the composition, including all ranges and subranges therebetween.

### Cyclodextrins

Compositions according to the disclosure optionally comprise at least one cyclodextrin, which includes derivatives thereof. Without being bound by theory, the use of at least one cyclodextrin may aid in preparing compositions having a uniform or substantially uniform viscosity.

Cyclodextrins are a family of cyclic oligosaccharides consisting of a macrocyclic ring of glucose subunits joined by α-1,4 glycosidic bonds. The cyclodextrins that can be used include those of the formula:

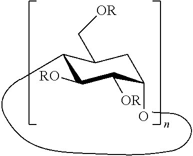

wherein:
R is chosen from H, CH₃, or a hydroxypropyl group, and
n ranges from 6-8.

For example, in embodiments where R═H, the cyclodextrin may be α-cyclodextrin (n=6), β-cyclodextrin (n=7), or γ-cyclodextrin (n=8). By way of example, α-cyclodextrin sold by the company WACKER under the name CAVAMAX W6 PHARMA, β-cyclodextrin sold by the company WACKER under the name CAVAMAX W7 PHARMA, or γ-cyclodextrin sold by the company WACKER under the name CAVAMAX W8 PHARMA can be used.

In other embodiments where R═CH₃, the cyclodextrin may be a methyl-cyclodextrin, such as methyl-α-cyclodextrin (n=6), methyl-β-cyclodextrin (n=7), or methyl-γ-cyclodextrin (n=8). For example, the methyl-β-cyclodextrin sold by the company WACKER under the name CAVASOL W7 may be chosen.

In various embodiments, the at least one cyclodextrin may comprise a mixture of cyclodextrins and/or derivatives thereof. For example, the at least one cyclodexctrin may be a mixture of α-cyclodextrin, β-cyclodextrin, and/or γ-cyclodextrin. In another embodiment, the at least one cyclodextrin includes β-cyclodextrin. In yet a further embodiment, the cyclodextrin is only β-cyclodextrin, and no other cyclodextrins or derivatives thereof are present in the composition.

If present, the at least one cyclodextrin may be included in the compositions in an amount ranging from about 0.01% to about 20% by weight, such as from about 0.1% to about 18%, from about 0.5% to about 15%, or from about 1% to about 13% by weight, relative to the total weight of the composition. By way of non-limiting example, the at least one cyclodextrin is present in the composition with a total amount ranging from about 0.5% to about 15%, by weight, for example, from about 0.5% to about 12%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 2% to about 15%, from about 2% to about 12%, from about 2% to about 10%, from about 2% to about 9%, from about 2% to about 8%, from about 2% to about 7%, from about 2% to about 6%, or from about 2% to about 5% by weight, relative to the total weight of the composition, including all ranges and subranges thereof.

### Additional Components

Compositions according to the disclosure may optionally comprise any additional or auxiliary component suitable for use in such compositions. Such components may include, but are not limited to, conditioning agents, moisturizing agents, fillers, structuring agents, shine agents, antioxidants (e.g. tetradibutyl pentaerithrityl hydroxyhydrocinnamate, butylated hydroxyanisole, and/or butylated hydroxytoluene), penetrants, sequestrants, fragrances, buffers (e.g. citric acid and/or sodium citrate), dispersants, plant extracts, preserving agents (preservatives), opacifiers, sunscreen agents, pH adjusting agents, vitamins (e.g. tocopherol, ascorbic acid, and/or ascorbyl glucoside), and antistatic agents.

Optional additional or auxiliary components may be present in an amount ranging up to about 15%, such as from about 0.01% to about 15%, about 0.1% to about 10%, or about 0.1% to about 5% by weight, relative to the total weight of the composition.

In various embodiments, the compositions may have a pH less than or equal to about 7, such as from about 3 to about 6.8, from about 3.5 to about 6.5, from about 4 to about 6.5, from about 4.5 to about 6.5, from about 5 to about 6.5 or from about 5.5 to about 6.5. In further embodiments, the compositions may have a pH of less than or equal to about 6, such as from about 2 to about 5, from about 2 to about 4, or from about 2 to about 3. In some embodiments, the pH of the compositions may be substantially unchanged (e.g. may have a change in pH of less than 20%, less than 15%, or less than 10%) when stored under ambient conditions for 4 weeks or more, such as 8 weeks.

In various embodiments, the compositions may have a viscosity ranging from about 900 mPa-s to about 3.5 Pa·s, such as about 1 Pa·s to about 3.3 Pa·s or about 2.5 Pa·s to about 3.2 Pa·s, when measured using a Rheomat RM 180, M3 spindle for 30 seconds under ambient conditions. In some embodiments, the viscosity of the compositions may be substantially unchanged (e.g. may have a change in viscosity of less than 20%, less than 15%, or less than 10%) when stored under ambient conditions for 3 days or more, such as 7 days or more, or 10 days or more.

### II. Methods

The present disclosure also relates to methods for styling hair, for example by temporarily straightening curly or wavy hair by application of the compositions described herein to the hair. The methods generally comprise applying any of the compositions according to the disclosure to the hair.

The compositions may be useful in a variety of settings, either for chemically treated or untreated hair, and/or for natural or unnatural curls. The degree of curliness or curl type of the hair may vary and is not limited. For example, the curls of hair been treated by the compositions disclosed herein may range from slightly wavy to very kinky and coily hair, and may have different textures and colors.

In addition to temporarily straightening the hair, the compositions are surprisingly capable of providing a variety of desirable sensory benefits, for example, moisture, smoothness, softness, shine, and/or anti-frizz properties, to the hair. As such, the compositions are useful in styling (which includes straightening) hair while also caring for hair, conditioning hair, and/or imparting one or more above-described sensory benefits to the hair.

Typically, methods according to the disclosure may include applying an effective amount of a composition disclosed herein, e.g. in the form of a cream emulsion, to hair having waves or curls, when the hair is dry, wet, damp, or moist. The hair may be either natural or synthetic hair. As used herein, the term “effective amount” refers to an amount sufficient to provide a desired straightening effect to the hair, taking into account the degree of curliness, the length, the volume, and the texture of the hair, and/or an amount sufficient to provide a desired sensory benefit to the hair. In general, from about 0.5 grams to about 50 grams of product is applied to the hair, depending on the specific product formulation, hair length, hair volume, and hair style type. The composition applied to the hair may, for example, be distributed evenly by combing through with fingers or a means such as a comb or the like. The composition may then be dried, for example air-dried or dried with a hair dryer. Heat may optionally be used during drying, but is not required. The composition may be allowed to remain on the hair as a leave-in product for any period of time as needed, for example, a few hours or a few days, or until the next washing or rinsing of the hair. Alternatively, the compositions may be rinsed from the hair after a brief leave-in period, from about a few seconds (e.g. 5, 10, 20, 30, or 60 seconds) to about 10, 20, or 30 minutes, or longer. In various embodiments, the composition does not form a noticeable (e.g. visual or tactile) film on the hair when it dries, which may be considered advantageous for consumers.

In some embodiments, before applying the composition to the hair, the hair may be first cleansed with a commercially available shampoo, and/or rinsed with water, and/or dried, either partially or completely. The composition may then be applied to the washed and/or rinsed hair when the hair is wet, damp, moist, or dry. In some other embodiments, before applying the composition to dry hair, the hair can optionally be moistened, damped, or wetted by water spray or using a wet towel, or by applying other treatment compositions that make the hair moist, damp, or wet.

It has surprisingly and unexpectedly been discovered that methods according to the disclosure generally do not require the use of a reducing agent, including a base, or heating the hair in order to achieve a temporary straightening effect. As such, in some embodiments, the method does not include using a reducing agent or a base, and/or does not including heating the hair.

In various embodiments, the methods of treating hair with the compositions according to the disclosure impart the benefits described above to the hair, without a greasy feel or flaking, relative to hair not having been treated with a composition according to the disclosure. The benefits imparted to the hair may remain a desired length of time, such as a few hours, a few days, or until the hair is rinsed or washed.

The compositions described herein are not mascara compositions, i.e. are not intended to be applied to eyelashes and/or eyebrows. The compositions are likewise not makeup compositions, i.e. are not intended to be applied to skin, in particular, to skin on the face, body, or lips. Thus, treatment of, or application to, the hair is intended to be limited to hair on the head, i.e. the scalp of the head, and not eyelashes, eyebrows, body or facial hair, or the like. In such embodiments, a skilled person understands that “hair on the head” means hair that grows from the scalp of the head, and excludes eyebrows, eyelashes, facial hair, etc.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, or F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, and a salt of F may be included. However, it is to be understood that such language also includes embodiments where G, H, I, etc., are present, as long as at least one of A, B, C, D, E, F, or a salt thereof, is also present.

The term “and/or” should be understood to include both the conjunctive and the disjunctive. For example, “A and/or B” means “A and B” as well as “A or B,” and expressly covers instances of either without reference to the other. For example, a composition that comprises “an emulsifier chosen from fatty alcohols and/or esters” means that the disclosure includes compositions comprising one or more fatty alcohols or one or more esters, as well as compositions comprising one or more fatty alcohols and one or more esters. As a further example, disclosure of “methods of styling the hair, straightening the hair, and/or reducing frizz of the hair” includes methods of styling the hair or methods of straightening the hair or methods of reducing frizz of the hair, as well as methods of styling the hair and straightening the hair, methods of styling the hair and reducing frizz of the hair, methods of styling the hair and straightening the hair and reducing frizz of the hair, etc.

As used herein, the phrases “and mixtures thereof,” “and a mixture thereof,” “and combinations thereof,” “and a combination thereof,” “or mixtures thereof,” “or a mixture thereof,” “or combinations thereof,” “or a combination thereof,” and variations thereof are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Further, unless expressly stated otherwise or when the context of the disclosure clearly dictates otherwise, the language “the at least one X may be present in an amount ranging from Y % to Z %” is to be understood to mean that the total amount of X's present in the composition ranges from Y % to Z %, inclusive of the endpoints. For example, “the at least one polyphenol may be present in an amount ranging from about 0.01% to about 10%” means that the total amount of polyphenols present ranges from about 0.01% to about 10%, inclusive of both 0.01% and 10%.

“Active material” as used herein with respect to the percent amount of an ingredient or raw material, refers to 100% activity of the ingredient or raw material.

All amounts given herein are relative to the amount of active material, unless otherwise indicated.

Unless otherwise indicated, all percentages herein are by weight, relative to the total weight of the composition.

All percentages, parts and ratios herein are based upon the total weight of the compositions of the present disclosure, unless otherwise indicated.

As used herein, the term “treat” (and its grammatical variations, such as “treating”) refers to the application of the compositions of the present disclosure onto the surface of the hair on the head.

As used herein, the phrase “applying a composition onto hair,” and variations thereof is intended to mean contacting the hair, with at least one of the compositions of the disclosure, in any manner. It may also mean contacting the hair in an effective amount.

As used herein, the term “styling” is intended to include “shaping” and “straightening.”

As used herein, the term “curly hair” refers to any hair including a curl, and “wavy hair” refers to any hair including a wave. The curl and/or wave may be natural or unnatural, i.e., formed by chemical treatment or physical treatment of the hair. The degree of curliness and/or waviness of the hair may vary and is not limited.

A “leave-in” composition or product refers to a hair-treatment composition that is not rinsed and/or washed away with water or acceptable solvent after the application of the composition onto the hair; instead, the composition is allowed to remain on the hair for a period of time as desired, such from 1 hour, 2 hours, 3 hours, 4 hours, up to 8 hours, overnight, or as long as needed, until next time of washing or rinsing the hair.

As used herein, the term “polyol” refers to an organic molecule comprising at least two free hydroxyl groups.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, unless expressly stated otherwise, the term “substantially free” or “essentially free” as used herein means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “salts” refers to throughout the disclosure may include salts having a counter-ion, in either ionized or un-ionized form. It is to be understood that, with regard to salts of acids described herein, it is intended to encompass the use of a salt of the acid as an ingredient added to a composition according to the disclosure, or to the salt of the acid that forms when the acid is used as an ingredient in a composition according to the disclosure (in ionized or un-ionized form).

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “temporary” should be understood to indicate that some degree of benefit or effect is imparted to the hair that remains from when the hair is treated with a composition according to the disclosure, until the composition is removed from the hair, e.g. by washing the hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition, unless otherwise noted.

### Example 1A—Compositions

The inventive and comparative compositions set forth in Table 1A-1 and 1A-2 below were prepared.

TABLE 1A-1
Inventive Compositions

| | 1A | 1B | 1C | 1D |
|---|---|---|---|---|
| CETRIMONIUM | 0.02 | 0.02 | 0.02 | 0.02 |
| CHLORIDE | | | | |
| TANNIC ACID | 2.0 | 2.0 | 2.0 | 2.0 |
| CETYL ESTERS | 1 | 1 | 1 | 1 |
| SCLEROTIUM GUM | 0.50 | | 0.50 | 0.50 |
| CETEARYL ALCOHOL | 5.0 | 5.0 | 5.0 | 5.0 |
| AMODIMETHICONE | 1.39 | 1.39 | 1.39 | 1.39 |
| BEHENTRIMONIUM | 0.79 | 0.79 | 0.79 | 0.79 |
| CHLORIDE | | | | |
| TRIDECETH-6 | 0.12 | 0.12 | 0.12 | 0.12 |
| OLEA EUROPAEA | 1.0 | 1.0 | | |
| (OLIVE) FRUIT OIL | | | | |
| COCONUT OIL | | | 3.0 | 3.0 |
| PVP | | 0.50 | | |
| GLUCOSE | | | 5.0 | |
| B-CYCLODEXTRIN | | | | 0.25 |
| ADDITIVES | 0.9 | 0.9 | 0.9 | 0.9 |
| (preservatives) | | | | |
| WATER | QS to 100 | QS to 100 | QS to 100 | QS to 100 |

TABLE 1A-2
Comparative Compositions

| | C1 | C2 | C3 |
|---|---|---|---|
| TANNIC ACID | | 2.0 | 2.0 |
| SCLEROTIUM GUM | | | 0.5 |
| CETYL ESTERS | 1.0 | 1.0 | |
| TRIDECETH-6 | 0.12 | 0.12 | |
| PYRIDOXINE HCl | 0.05 | | |
| NIACINAMIDE | 0.05 | | |
| BEHENTRIMONIUM CHLORIDE | 0.79 | 0.79 | |
| ARGANIA SPINOSA KERNEL OIL | 0.10 | | |
| CETRIMONIUM CHLORIDE | 0.02 | 0.02 | |
| CAPRYLYL GLYCOL | <0.01 | | |
| CHLORHEXIDINE | 0.03 | | |
| DIHYDROCHLORIDE | | | |
| CETEARYL ALCOHOL | 5.0 | 5.0 | |
| AMODIMETHICONE | 1.39 | 1.39 | |
| OLEA EUROPAEA (OLIVE) | | 1.0 | |
| FRUIT OIL | | | |
| SACCHARUM OFFICINARUM | 0.02 | | |
| (SUGARCANE) EXTRACT | | | |
| ADDITIVES (pH adjusters, | 1.0 | 0.9 | |
| preservatives, dyes, | | | |
| fragrances, plant extracts, | | | |
| humectants, vitamins) | | | |
| WATER | QS to 100 | QS to 100 | QS to 100 |

The compositions were prepared as follows. First, the solvent phase was heated to a temperature of about 70-75° C. The gum, polymer, and polyphenol components were added individually with mixing using a rotor-stator blade on a Rayneri V2004 mixer. Once the polyphenol, polymers, and gum were fully dispersed, the rest of the ingredients were added one by one with mixing at medium speed to ensure each was fully incorporated, and an emulsion was formed. Then the batch was cooled to a temperature of about 30-35° C., while mixing with a propeller-type blade.

Each of compositions 1A-1D and C1-C3 provided temporary straightening benefits to hair, and reduced frizz even in high humidity. However, hair treated with compositions C1-C3 was more stiff and coated than hair treated with compositions 1A-1D, which had a smoother, more natural feel.

### Example 1B—Compositions

The inventive and comparative compositions set forth in Table 1B below were prepared following the procedure of Example 1A.

TABLE 1B

| | Inventive | Comparative |
|---|---|---|
| | 1E | C4 |
| TANNIC ACID | 2.0 | 2.0 |
| SCLEROTIUM GUM | 0.5 | |
| CARRAGEENAN | | 0.5 |
| CETYL ESTERS | 1.0 | 1.0 |
| TRIDECETH-6 | 0.12 | 0.12 |
| ISOPROPYL ALCOHOL | 0.18 | 0.18 |
| BEHENTRIMONIUM CHLORIDE | 0.79 | 0.79 |
| CETRIMONIUM CHLORIDE | 0.02 | 0.02 |
| HEXYLENE GLYCOL | 0.5 | 0.5 |
| CETEARYL ALCOHOL | 5.0 | 5.0 |
| AMODIMETHICONE | 1.39 | 1.39 |
| COCONUT OIL | 3.0 | 3.0 |
| B-CYCLODEXTRIN | 1.0 | 1.0 |
| WATER | QS to 100 | QS to 100 |

### Example 2A—Stability Testing

The stability of inventive compositions 1A and 1B was evaluated by measuring the pH and viscosity, and by visual appearance of the compositions, over 10 days.

TABLE 2A
pH

| | Day 0 | Day 1 | | | Day 2 | | | Day 6 | | |
|---|---|---|---|---|---|---|---|---|---|---|
| Comp. | pH | T | pH | Δ pH | T | pH | Δ pH | T | pH | Δ pH |
| 1A | 2.65 | 25° C. | 2.73 | 0.08 | 25° C. | 2.90 | 0.25 | 25° C. | 2.76 | 0.11 |
| | | 60° C. | 2.82 | 0.17 | 60° C. | 2.86 | 0.21 | 60° C. | 2.93 | 0.28 |
| 1B | 2.33 | 25° C. | 2.73 | 0.40 | 25° C. | 2.82 | 0.49 | 25° C. | 3.10 | 0.77 |
| | | 60° C. | 2.83 | 0.50 | 60° C. | 2.88 | 0.55 | 60° C. | 2.90 | 0.57 |

The pH of compositions 1A and 1B increased slightly over time at both room temperature and elevated temperature, but stabilized by the second day. Thus, the pH data confirms the inventive compositions are stable.

TABLE 2B
Viscosity

| | Day 0 | Day 1 | | | Day 2 | | | Day 6 | | |
|---|---|---|---|---|---|---|---|---|---|---|
| | η | | η | Δ | | η | Δ | | η | Δ |
| Comp. | (Pa · s) | T | (Pa · s) | η | T | (Pa · s) | η | T | (Pa · s) | η |
| 1A | 1.1 | 25° C. | 2.2 | 1.1 | 25° C. | 2.6 | 1.5 | 25° C. | 3.0 | 1.9 |
| | | 60° C. | 1.4 | 0.3 | 60° C. | 1.6 | 0.5 | 60° C. | 1.5 | 0.4 |
| 1B | 1.2 | 25° C. | 1.8 | 0.6 | 25° C. | 2.5 | 1.3 | 25° C. | 3.2 | 2.0 |
| | | 60° C. | 2.6 | 1.4 | 60° C. | 2.4 | 1.2 | 60° C. | 1.7 | 0.5 |

The viscosity of compositions 1A and 1B was measured using a Rheomat RM 180, M3 spindle for 30 seconds under ambient conditions (i.e. room temperature and pressure). The viscosity of the compositions decreased initially upon preparation, but stabilized by the second day. Thus, the viscosity data confirms that the inventive compositions are stable.

In addition, compositions 1A and 1B were evaluated visually over the 10-day period. No solid precipitate was visible either in either solution or at the bottom of the container by day 10, further confirming the stability of the compositions.

### Example 2B—Stability Testing

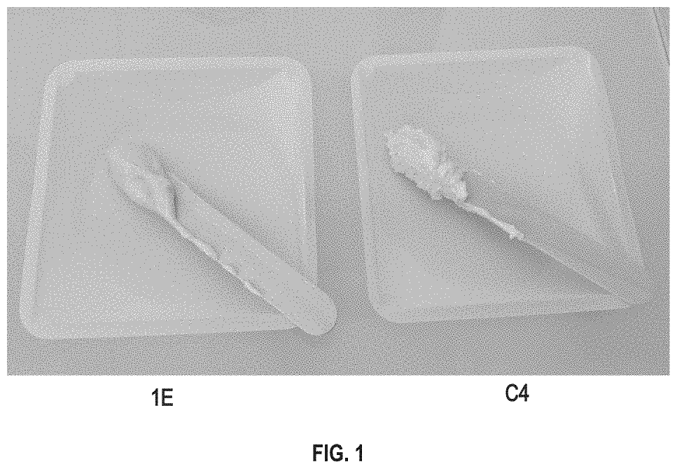

Compositions 1E and C4 were evaluated visually upon their preparation. Composition 1E, which comprises a nonionic polymer, was smooth, homogenous, and stable. In contrast, composition C4, which does not have a nonionic polymer but rather contains carrageenan, which is anionic, was not smooth, was inhomogenous and unstable. FIG. 1 is a photograph where the difference in the quality of the two compositions is easily discernible.

### Example 3A—Testing on Hair

Compositions 1C and C1 were tested on a mannequin head with curly hair. First, the hair was washed and conditioned with a standard shampoo and conditioner, and then excess water was removed with a towel. Next, equal amounts of composition 1C or composition C1 was applied to one-half of the mannequin head in an amount of 3.5 grams per half-head, and distributed through the hair by hand, after which the hair was dried with a rollerball dryer at a temperature of about 40-45° C. for a period of about 60 minutes.

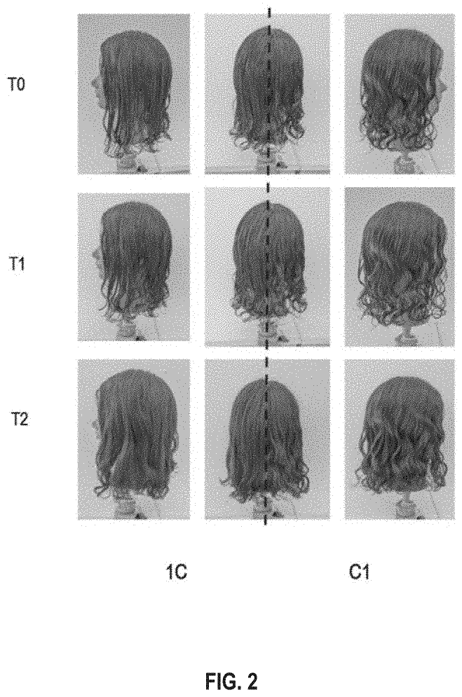

FIG. 2 shows side and back views of the hair after either composition 1C (left half-head) or C1 (right half-head) is applied and while the hair is wet (TO), once the hair is dried (T1), and after the hair has been touched (T2). As seen in the figure, inventive composition 1C provides straightening benefits to curly hair, in contrast to the hair treated with comparative composition C1. The hair treated with composition 1C did not appear visually to have a film after the hair dried, and no film was detected when the hair was touched. The hair treated with both compositions had good sensorial feel.

### Example 3B—Testing on Hair

Compositions 1D and C2 were tested on a mannequin head with curly hair. First, the hair was washed and conditioned with a standard shampoo and conditioner, and then excess water was removed with a towel. Next, equal amounts of composition 1D or composition C2 was applied to one-half of the mannequin head in an amount of 3.5 grams per half-head, and distributed through the hair by hand, after which the hair was dried with a rollerball dryer at a temperature of about 40-45° C. for a period of about 60 minutes.

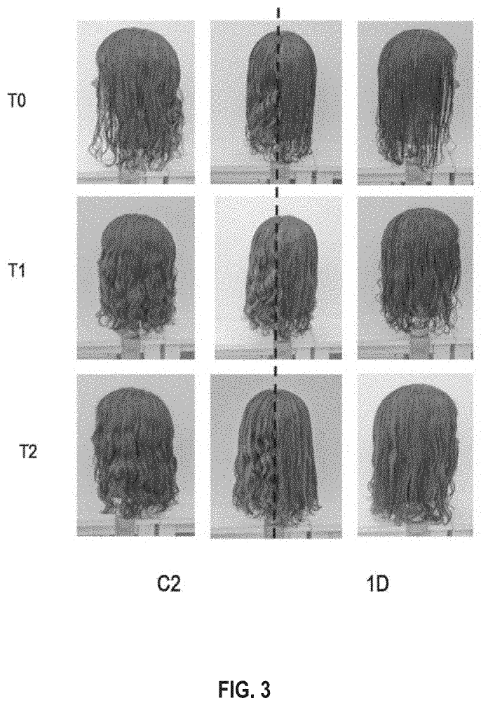

FIG. 3 shows side and back views of the hair after either composition 1D (right half-head) or C2 (left half-head) is applied and while the hair is wet (TO), once the hair is dried (T1), and after the hair has been touched (T2). As seen in the figure, inventive composition 1D provides straightening benefits to curly hair, superior to those imparted to the hair treated with comparative composition C2. The hair treated with composition 1D did not appear visually to have a film after the hair dried, and no film was detected when the hair was touched. The hair treated with both compositions had good sensorial feel.

### Example 3C—Testing on Hair

Compositions 1A and C3 were tested on a mannequin head with curly hair. First, the hair was washed and conditioned with a standard shampoo and conditioner, and then excess water was removed with a towel. Next, equal amounts of composition 1A or composition C3 was applied to one-half of the mannequin head in an amount of 3.5 grams per half-head, and distributed through the hair by hand, after which the hair was dried with a rollerball dryer at a temperature of about 40-45° C. for a period of about 60 minutes.

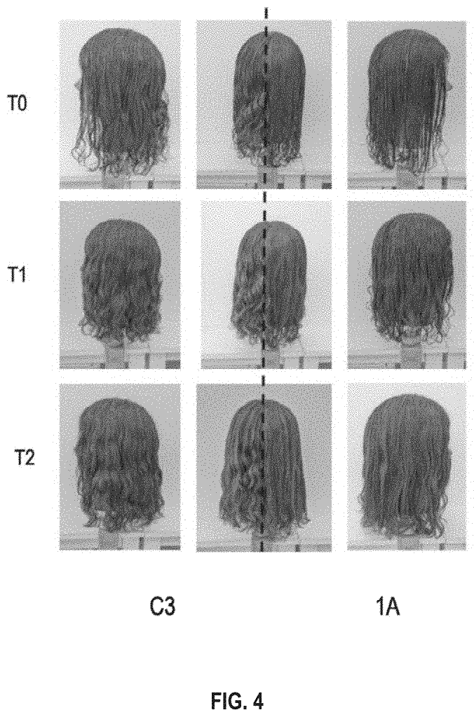

FIG. 4 shows side and back views of the hair after either composition 1A (right half-head) or C3 (left half-head) is applied and while the hair is wet (T0), once the hair is dried (T1), and after the hair has been touched (T2). As seen in the figure, the hair treated with both compositions 1A and C3 demonstrated straightening of the hair. However, although the hair treated with composition 1A did not appear visually to have a film after the hair dried, and no film was detected when the hair was touched, the hair treated with composition C3 had a rigid film once the hair dried that had to be broken for a natural look and feel. The hair treated with both compositions had good sensorial feel after the film on the hair treated with composition C3 was broken.

The above Examples demonstrate that compositions according to the disclosure provide effective temporary straightening benefits to curly hair, while at the same time providing sensorial benefits such as smoothness and frizz-control to the hair. Further, compositions according to the disclosure have improved stability compared to compositions not according to the disclosure.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-40. (canceled)

41. A hair styling composition comprising:
(a) at least one polyphenol;
(b) at least one nonionic polysaccharide polymer;
(c) at least one cationic surfactant;
(d) at least one emulsifier chosen from fatty alcohols, esters, or mixtures thereof;
(e) at least one solvent comprising water;
(f) optionally, at least one silicone compound; and
(g) optionally, at least one oil.

42. The hair styling composition of claim 41, wherein the total amount of polyphenols ranges from about 0.01% to about 10% by weight, relative to the total weight of the composition.

43. The hair styling composition of claim 41, wherein the at least one polyphenol comprises tannic acid.

44. The hair styling composition of claim 41, wherein the at least one nonionic polysaccharide polymer is chosen from nonionic C1-C3 alkyl guars, nonionic C1-C3 hydroxyalkyl guars, glucans, modified or non-modified starches, dextrans, celluloses, cellulose derivatives, or mixtures thereof.

45. The hair styling composition of claim 41, wherein the total amount of nonionic polysaccharide polymers ranges from about 0.01% to about 5% by weight, relative to the total weight of the composition.

46. The hair styling composition of claim 41, wherein the at least one cationic surfactant comprises at least one quaternary ammonium salt of formula (I): 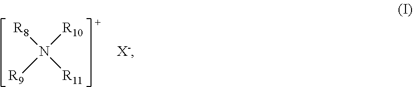
wherein:
R8 to R11 are independently chosen from linear or branched aliphatic groups containing from 1 to 30 carbon atoms, or an aromatic group, with the proviso that at least one of the groups R8 to R11 includes from 8 to 30 carbon atoms; and
X⁻ is an anion chosen from bromides, chlorides, iodides, fluorides, phosphates, acetates, lactates, (C1-C4)alkyl sulfates, (C1-C4)alkyl sulfonates or (C1-C4)alkylaryl sulfonates; C1-C30 alkyl, C1-C30 alkoxy, (C2-C6)polyoxyalkylene, C1-C30 alkylamide, (C12-C22)alkyl-(C2C6)alkylamido, (C12-C22)alkyl acetate, and C1-C30 hydroxyalkyl groups.

47. The hair styling composition of claim 41, wherein the total amount of cationic surfactants ranges from about 0.01% to about 10% by weight, relative to the total weight of the composition.

48. The hair styling composition of claim 41, wherein the at least one emulsifier comprises at least one fatty alcohol and at least one ester.

49. The hair styling composition of claim 48, wherein the at least one fatty alcohol is chosen from decyl alcohol, undecyl alcohol, dodecyl alcohol, myristyl alcohol, cetyl alcohol, stearyl alcohol, cetearyl alcohol, isostearyl alcohol, isocetyl alcohol, behenyl alcohol, linalool, oleyl alcohol, myricyl alcohol, or mixtures thereof.

50. The hair styling composition of claim 48, wherein the at least one ester is chosen from isopropyl esters, cetyl esters, glyceryl esters, or a mixture thereof.

51. The hair styling composition of claim 41, comprising at least one silicone compound chosen from silicone acrylate polymers, hydroxylmethylpolysiloxane, amodimethicones, dimethicone copolyols, polydimethylsiloxanes, polydiethylsiloxanes, polydimethyl siloxanes having terminal hydroxyl groups, or mixtures thereof.

52. The hair styling composition of claim 41, comprising at least one silicone compound, wherein the total amount of silicone compounds ranges from about 0.1% to about 10% by weight, relative to the total weight of the composition.

53. The hair styling composition of claim 41, further comprising at least one cyclodextrin.

54. The hair styling composition of claim 41, wherein the composition comprises water in an amount of at least about 50% by weight, relative to the total weight of the composition.

55. The hair styling composition of claim 41, wherein the solvent comprises water and at least one non-aqueous solvent, wherein the non-aqueous solvent is present in an amount less than about 20% by weight, relative to the total weight of the composition.

56. A hair styling composition comprising:
(a) tannic acid;
(b) at least one nonionic polysaccharide polymer;
(c) at least one cationic surfactant;
(d) at least one emulsifier chosen from fatty alcohols, esters, or mixtures thereof;
(e) water, present in an amount of at least 50% by weight, relative to the total weight of the composition;
(f) at least one amino functional silicone; and
(g) optionally, at least one oil.

57. A method for styling hair comprising applying to the hair a hair styling composition, wherein the hair styling composition comprises:
(a) at least one polyphenol;
(b) at least one nonionic polysaccharide polymer;
(c) at least one cationic surfactant;
(d) at least one emulsifier chosen from fatty alcohols, esters, or mixtures thereof;
(e) at least one solvent comprising water;
(f) optionally, at least one silicone compound; and
(g) optionally, at least one oil.

58. The method of styling hair according to claim 57, wherein the composition is left on the hair until the hair is washed.

59. The method of styling hair according to claim 57, wherein the composition applied to the hair does not comprise a reducing agent.

60. The method of styling hair according to claim 57, wherein no step of heating the hair occurs after the composition is applied to the hair.
